(19) United States
(12) Patent Application Publication
(10) Pub. No.: US 2011/0152897 A1
(43) Pub. Date: Jun. 23, 2011

(54) HERNIA PATCH WITH REMOVABLE RESILIENT ELEMENT

(76) Inventor: Brian L. Bates, Bloomington, IN (US)

(21) Appl. No.: 13/029,347

(22) Filed: Feb. 17, 2011

Related U.S. Application Data
(63) Continuation of application No. PCT/US2009/055171, filed on Aug. 27, 2009.
(60) Provisional application No. 61/093,735, filed on Sep. 3, 2008.

(51) Int. Cl. A61B 17/00

(52) U.S. Cl. 606/151

(57) ABSTRACT

The invention provides, in certain aspects, grafting devices deliverable into the body for repairing defects in bodily structure walls. One such grafting device comprises a compliant sheet-form material, and a removable resilient element that is retained in association with the sheet-form material. In some forms, the resilient element is adapted for delivery in its entirety into the body, and thereafter, can be disassociated from the sheet-form material for removal from the body. The sheet-form material may be formed with one or more of a variety of biocompatible materials including some that are naturally derived and some that are non-naturally derived. Illustratively, the sheet-form material may be comprised of a remodelable, angiogenic material, for example, a remodelable extracellular matrix (ECM) material. In additional embodiments, the invention provides methods and apparatuses for delivering these and other inventive grafting device into the body.

### CROSS-REFERENCE TO RELATED APPLICATIONS

This application is a continuation of International Application No. PCT/US2009/055171, filed Aug. 27, 2009, which claims the benefit of U.S. Provisional Application No. 61/093,735, filed Sep. 3, 2008, each of which is hereby incorporated by reference.

### BACKGROUND

The present invention relates generally to medical devices and in particular aspects to devices for repairing defects in bodily structure walls.

As further background, it is estimated that tens of millions of people throughout the world develop hernias each year. Men and women of all ages can have hernias. A hernia is essentially an opening in the abdominal wall through which abdominal contents such as bowels may protrude. A hernia occurs when the inside layers of the abdominal wall weaken and then bulge or tear. The inner lining of the abdomen pushes through the weakened area to form a balloon-like sac. This, in turn, can cause a loop of intestine or abdominal tissue to slip into the sac, causing pain and other potentially serious health problems. Hernias usually occur either because of a natural weakness in the abdominal wall or from excessive strain on the abdominal wall, such as the strain from heavy lifting, substantial weight gain, persistent coughing, or difficulty with bowel movements or urination.

Approximately eighty percent of all hernias are located near the groin. Hernias may also occur below the groin (femoral), through the navel (umbilical), and along a previous incision (incisional or ventral). Inguinal or groin hernias can occur in the weakened wall or inguinal floor of the abdomen in Hesselbach's triangle. This type of hernia is called a direct hernia. An indirect hernia occurs at the internal ring adjacent to the vas deferens as it exits the abdomen to become part of the spermatic cord.

All hernias represent a potentially life-threatening condition. Once a hernia is diagnosed, it should be repaired unless there is some contraindication. Hernias usually need to be surgically repaired to prevent intestinal damage and further complications. A variety of surgical methods have been developed for treating hernias including several different “open” surgical methods, as well as methods that are considered less invasive (e.g., laparoscopic methods). Although open hernia surgery is still common, it is undesirably lengthy, and therefore, costly. Open surgery also requires a large incision with excessive dissection of normal tissue, causes excessive pain and discomfort to the patient, involves unacceptably long recovery and work disability time, and results in an unacceptably high recurrence rate.

There remain needs for improved and/or alternative devices and methods for repairing hernias and other bodily structure wall defects. The present invention is addressed to those needs.

### SUMMARY

The present invention provides, in certain aspects, unique apparatuses for delivering grafting devices into the body. One such apparatus comprises a delivery device having a lumen communicating with a distal end opening, and a grafting device positioned in the delivery device lumen. The delivery device distal end opening is configured for passage into the body. In some cases, the delivery device is a laparoscope or other similar device. The grafting device is effective to repair a defect in a wall of a bodily structure, and is comprised of a compliant sheet-form material and a removable resilient element retained in association with the sheet-form material. The resilient element is adapted for delivery in its entirety into the body and for disassociation from the sheet-form material after the grafting device is delivered into the body. The resilient element exhibits a deformed first condition when the grafting device is positioned in the delivery device lumen, and is adapted to attain a second (e.g., generally relaxed) condition when the grafting device is removed from the delivery device lumen. This relaxed second condition is effective to present at least a segment of the sheet-form material in a generally planar form in the body for placement at the bodily structure wall defect. In some embodiments, an inventive apparatus of this sort further comprises a pushing member positioned in the delivery device lumen. Such a pushing member is translatable in the delivery device lumen, and is effective to push the grafting device out of the delivery device lumen through the distal end opening.

In another embodiment, the invention provides a method for delivering a grafting device into the body, which utilizes an apparatus such as that described above. In one step, the delivery device distal end opening is positioned in the body. The grafting device is then removed from the delivery device lumen through the distal end opening, wherein the resilient element is delivered in its entirety into the body, and attains a second condition effective to present at least a segment of the sheet-form material in a generally planar form for placement at the bodily structure wall defect. The sheet-form material can then be positioned over the bodily structure wall defect, and anchored to the body to maintain the sheet-form material over the bodily structure wall defect. In another step, the resilient element can be disassociated from the sheet-form material for removal from the body. In some cases, the bodily structure wall defect includes herniated tissue. Additionally, anchoring the sheet-form material to the body can include anchoring the sheet-form material to the bodily structure wall. The material can be anchored in a variety of manners, for example, by methods that involve fastening and/or bonding the material to a bodily structure.

A further aspect of the present invention provides a grafting device deliverable into the body for repairing a defect in a wall of a bodily structure. This grafting device comprises a compliant sheet-form material, and a removable resilient element that is retained in association with the sheet-form material and exhibits a relaxed condition effective to present at least a segment of the sheet-form material in a generally planar form. The resilient element is adapted for delivery in its entirety into the body, and has a retrieving portion that extends from the sheet-form material. The retrieving portion is adapted for retrieval in the body for disassociating the resilient element from the sheet-form material for removal from the body. The sheet-form material can exhibit a variety of shapes and sizes, and may be formed with one or more of a variety of biocompatible materials including some that are naturally derived and some that are non-naturally derived. In a preferred embodiment, the sheet-form material is comprised of a remodelable, angiogenic material, for example, a remodelable extracellular matrix material such as submucosa. A resilient element of this sort can have numerous shapes and sizes, and may be formed with one or more of a variety of materials, whether occurring as a single- or multiple-piece arrangement. In one form, the resilient element includes one or more pieces of Nitinol or other similar wire. As well, the resilient element may be retained in association with the sheet-form material in any suitable manner. Illustratively, a receiving area in which the resilient element can be received for retaining the resilient element in association with the sheet-form material may occur along the material. In one embodiment, such a receiving area is comprised of a folded peripheral region of the sheet-form material. Additionally or alternatively, an inventive device may include a retaining adaptation bonded or coupled to or otherwise joined with the sheet-form material for retaining the resilient element in association with the sheet-form material.

Other objects, embodiments, forms, features, advantages, aspects, and benefits of the present invention shall become apparent from the detailed description and drawings included herein.

### BRIEF DESCRIPTION OF THE DRAWINGS

FIG. 1 is a perspective view of a grafting device according to one embodiment of the present invention.

FIG. 2 shows the grafting device of FIG. 1 in a partially rolled configuration.

FIG. 3 is a perspective view of an apparatus of the present invention that includes the grafting device of FIG. 1 positioned in a delivery device lumen.

FIG. 4 is a top view of another grafting device of the present invention.

FIG. 5 is a partial, top view of a grafting device according to another embodiment of the present invention.

FIG. 6 is a top view of another grafting device of the present invention.

FIG. 7 is a top view of an additional grafting device of the present invention.

FIG. 8 is a top view of a grafting device according to another embodiment of the present invention.

FIG. 9 is a top view of yet another grafting device of the present invention.

### DETAILED DESCRIPTION

While the present invention may be embodied in many different forms, for the purpose of promoting an understanding of the principles of the present invention, reference will now be made to the embodiments illustrated in the drawings, and specific language will be used to describe the same. It will nevertheless be understood that no limitation of the scope of the invention is thereby intended. Any alterations and further modifications in the described embodiments and any further applications of the principles of the present invention as described herein are contemplated as would normally occur to one skilled in the art to which the invention relates.

As disclosed above, in certain aspects, the present invention provides unique grafting devices for repairing defects in bodily structure walls. One such grafting device comprises a compliant sheet-form material, and a removable resilient element retained in association with the sheet-form material. The resilient element is deformable, and when in a non-deformed or “relaxed” condition, is effective to present at least a segment of the associated sheet-form material in a generally planar form. When deformed, for example, when the shape of the grafting device of which it is a part is somehow transformed (e.g., by rolling and/or folding, etc.), the resilient element is then poised to essentially return to its non-deformed condition and again present the associated sheet-form material in a generally planar form. In some embodiments, the resilient element is compactable to a compacted, first condition, and when in this compacted condition, is then expandable to an expanded, second condition. In forms where a deformed resilient element has the capacity to expand, these resilient elements can include those that are considered self-expanding and those that require at least some manipulation in order to expand. The resilient element is adapted for delivery in its entirety into the body, and in some forms, has a retrieving portion that is configured to extend a distance from the sheet-form material The retrieving portion is adapted for retrieval in the body for disassociating the resilient element from the sheet-form material for removal from the body. In one embodiment, a grafting device of this sort is a hernial repair patch.

Additionally, the present invention provides apparatuses for delivering these and other inventive grafting devices into the body. One such apparatus comprises a delivery device having a lumen communicating with a distal end opening, and a grafting device such as that described above positioned in the delivery device lumen. Thus, when the grafting device is positioned in the delivery device lumen, the resilient element is deformed in some manner along with the compliant sheet-form material. Then, when the grafting device is removed from the delivery device lumen, the resilient element can return to its non-deformed condition and again present at least a segment of the associated sheet-form material in a generally planar form. Optionally, such an apparatus includes a pushing member positioned in the delivery device lumen. This pushing member is translatable in the delivery device lumen, and is effective to push the grafting device out of the delivery device lumen through its distal end opening. In one embodiment, a delivery device of this sort is a laparoscope or other similar device.

The invention also provides methods for delivering grafting devices into the body. In one inventive method, an apparatus such as that described above is provided, and the delivery device distal end opening is positioned in the body. The grafting device is then removed from the delivery device lumen through the distal end opening, wherein the resilient element is delivered in its entirety into the body. Upon removal, the once-constrained resilient element is able to at least partially return to its relaxed or non-deformed condition, wherein it is effective to present at least a segment of the sheet-form material in a generally planar form for placement at the bodily structure wall defect. The sheet-form material can then be positioned over the bodily structure wall defect, and anchored within the body to maintain the sheet-form material over the bodily structure wall defect. The resilient element can then be disassociated from the sheet-form material and removed from the body. In some cases, a grafting device will be delivered to a relatively confined space in the body such that the resilient element will not be able to return to a substantially non-deformed condition, at least not without some additional manipulation. In such instances, if a different amount of resilient element deformation is desired following initial placement, the grafting device may be repositioned or otherwise manipulated in the body to achieve the desired amount.

The devices described herein have broad application. In certain aspects, inventive devices are useful in procedures to replace, augment, support, repair, and/or otherwise suitably treat diseased or otherwise damaged or defective patient tissue. Thus, while some of the devices described herein are useful in treating herniated tissue, inventive devices can be used to treat non-herniated tissue as well. In this regard, devices of the invention can be used in any procedure where the application of a graft material to a bodily structure can provide benefit to the patient.

Further in this regard, the grafting devices described herein can be delivered into the body in a variety of manners. Illustratively, the delivery of a device may involve a laparoscope or other similar delivery instrument. In some forms, an inventive apparatus includes a delivery instrument that can effectively maintain a compactable graft device in a compacted condition for passage into the body. Then, when the compacted device has been desirably passed into the body with the instrument, the graft device can be released or otherwise disassociated from the delivery device where it can at least partially return to a non-compacted condition. Although not necessary to broader aspects of the invention, in some cases, an instrument of this sort includes a wall portion configured to wholly or partially surround the compacted graft device and maintain the device in this compacted condition for a more low-profile delivery into the body. These and other adaptations for maintaining a graft device in a compressed or otherwise compacted condition for delivery into the body will be recognized by the skilled artisan and are therefore encompassed by the present invention.

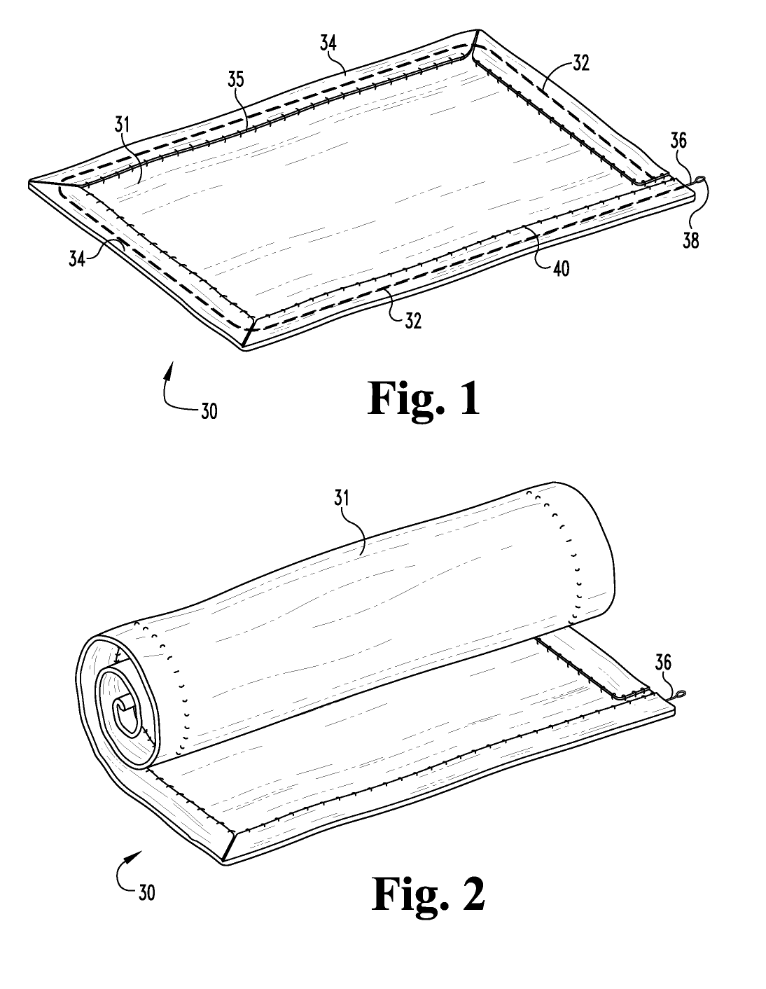

Referring now to FIG. 1, shown is a grafting device 30 according to the present invention. Device 30 includes a piece of compliant sheet-form material 31 and a resilient element 32. Material piece 31 exhibits a generally rectangular shape, and may be formed with one or more of a variety of materials including some that are naturally derived and some that are non-naturally derived as discussed more thoroughly below. Resilient element 32 is removably positioned in a receiving area 34 occurring along the periphery of material piece 31. When so positioned, resilient element 32 is effective to present sheet-form material 31 in a generally planar form as shown in FIG. 1. In this particular embodiment, outer edges 35 of the piece of material are folded over and sutured to form a sleeve or sleeve-like receiving area. Such a sleeve can be formed around the resilient element, or alternatively, the sleeve can be formed and then the resilient element positioned therein. Resilient element 32 may also vary as to materials of construction. In some preferred embodiments, such a resilient element is a single piece of Nitinol wire having a plurality of sides and bends.

Although not necessary to broader aspects of the invention, in some cases, a resilient element includes a portion extending a distance away from the sheet-form material to facilitate disassociation of the resilient element from the remainder of the device once it is inside the body. For example and referring again to FIG. 1, resilient element 32 includes a retrieving portion 36. Retrieving portion 36 extends a distance from sheet-form material 31, and is adapted for retrieval in the body for disassociating the resilient element from the sheet-form material for removal from the body. In this particular embodiment, retrieving portion 36 includes a looped tip 38, which can facilitate retrieval of the retrieving portion inside the body.

While resilient element 32, when in a relaxed condition, is effective to present sheet-form material 31 in a generally planar form, compliant sheet-form material 31 and resilient element 32 are such that grafting device 30 can be transformed into a variety of other shapes. The shape of an inventive device such as device 30 can be altered in any suitable manner including some that involve folding, rolling and/or otherwise suitably deforming the device. For example and referring now to FIG. 2, device 30 can be rolled into a generally cylindrical form. In this “deformed” configuration, resilient element 32 is poised to return to a “non-deformed” configuration (i.e., unroll) to again present sheet-form material 31 in a generally planar form. In some instances, a grafting device of the invention is deformed so as to be able to position the device in a delivery device lumen.

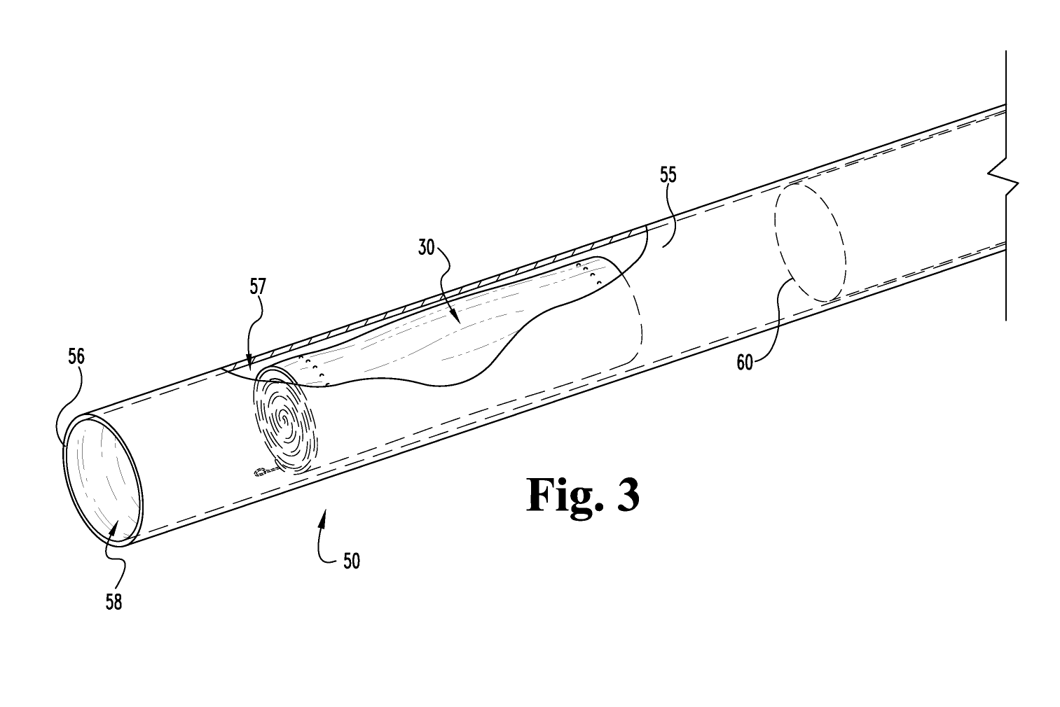

With reference now to FIG. 3, shown is an apparatus 50 for delivering a grafting device such as device 30 into the body. Apparatus 50 includes a delivery device 55 having a distal end 56. Delivery device 55 also includes a lumen 57 communicating with a distal end opening 58. As shown in FIG. 3, grafting device 30 can be fully rolled and positioned in delivery device lumen 57. In the current embodiment, an optional pushing member 60 is positioned in lumen 57. Pushing member 60 is translatable in the lumen, and is effective to push grafting device 30 out of the delivery device lumen through distal end opening 58.

In one method of use, the delivery device distal end 56 is positioned in the body with grafting device 30 positioned in delivery device lumen 57. Thereafter, grafting device 30 is removed from the delivery device lumen through distal end opening 58 so that the resilient element 32 is delivered in its entirety into the body. Once removed from the delivery device lumen, resilient element 32 unrolls to present all or part of sheet-form material 31 in a generally planar form in the body. The sheet-form material is then positioned over a bodily structure wall defect and anchored to the body to maintain the sheet-form material over the defect. Thereafter, the operator grasps the retrieving portion to disassociate the resilient element from the sheet-form material and remove it from the body.

Delivery devices useful in certain aspects of the present invention have a lumen communicating with a distal, open end. This “leading” distal end is configured to pass into the body. Although not necessary to broader aspects of the invention, this distal end, or any portion thereof, may be particularly configured to enhance travel of the device through certain portions of the body, for example, including a tapered portion and/or having a dome-shaped or otherwise rounded tip. Accordingly, such devices can exhibit any suitable size, shape and configuration for performing the functions described herein.

In some embodiments, a delivery device is rigid or substantially rigid, and is configured to be generally straight. Alternatively, delivery devices useful in the invention can be configured to include one or more portions that are curvilinear, bent, or otherwise suitably shaped. In certain aspects, the distal end of a delivery device is curved to a degree to allow for easier passage of the distal end into certain body regions. In some forms, a delivery device is composed of a malleable material such as but not limited to a woven or spirally-configured metal or alloy material, or a plastic (hydrocarbon-based) material, which may be bent to a necessary angle or curvature for passage into certain body spaces. The shape of such a delivery device may be adjusted at certain intervals of the procedure so as to allow the delivery device to pass further and further into the body. In some forms, the delivery device is generally straight in a relaxed condition but can flex to adapt to contours during passage.

In this regard, delivery devices, when used in the invention, can be formed with one or more of a variety of materials. A particular material may be selected to take advantage of one or more of its properties such as but not limited to its weight, durability, flexibility, etc. For example, a device may comprise a material having properties that allow the device to traverse a volume of tissue or other body space without buckling or kinking or causing unacceptable damage to surrounding soft tissues and/or other body parts. Illustratively, the device, or selected portions thereof (e.g., the distal end), can exhibit a degree of flexibility. In this regard, a delivery device, or any portion thereof, may be rigid, malleable, semi-flexible, or flexible. In certain embodiments, an advanceable device is particularly adapted for moving through and into body regions where the path taken angulates sharply or curves abruptly. In some of these embodiments, the device is configured to be directable or steerable through the body, and therefore, exhibits desirable characteristics, e.g., sufficient stiffness, to allow an operator to apply an adequate degree of ante-grade force to the device to allow it to traverse a bodily region in a desirable manner.

Suitable materials for forming delivery devices or device components of the invention can include but are not limited to metallic materials including stainless steel, titanium, cobalt, tantalum, gold, platinum, nickel, iron, copper and the like, as well as alloys of these metals (e.g., cobalt alloys, such as Elgiloy®, a cobalt-chromium-nickel alloy, MP35N, a nickel-cobalt-chromium-molybdenum alloy, and Nitinol®, a nickel-titanium alloy). Additionally or alternatively, the delivery device can include material in the form of yarns, fibers, and/or resins, e.g., monofilament yarns, high tenacity polyester, and the like. A delivery device can also include other plastic, resin, polymer, woven, and fabric surgical materials, other conventional synthetic surgical materials, such as a shape-memory plastic, and/or combinations of such materials. Further, appropriate ceramics can be used, including, without limitation, hydroxyapatite, alumina and pyrolytic carbon.

In some forms, a flexible delivery device will incorporate one or more adaptations for facilitating removal of the device from the body during a delivery procedure. Illustratively, a delivery device wall can incorporate scores, thinner portions, and other openings and non-openings that weaken a portion of the wall to facilitate a tear-away operation in removing the device from the body. Such a weakened portion may include any suitable means for facilitating tearing or breaking along the area. In certain beneficial forms, a delivery sleeve or other similar device is controllably separable longitudinally into two or more pieces for removal, for example, as occurs in Peel-Away® catheters available from Cook Incorporated, Bloomington, Ind., USA. Such an apparatus with a separable sleeve is particularly useful in treating internal bodily structures that are relatively difficult to access.

Turning now to a more detailed discussion of compliant sheet-form materials useful in the invention, an inventive device can incorporate one or more individual pieces of compliant material. Although not necessary to broader aspects of the invention, when a device includes multiple material pieces, any given piece of material may be attached to any other piece of material present in the device. Material pieces can be attached to one another or otherwise joined in a variety of manners including some that involve bonding the pieces together with a bonding agent and some that involve coupling the pieces together with sutures, staples and/or other objects known in the art for combining pieces of material. As well, two material pieces can be joined together at one or more of a variety of locations along the respective pieces. Illustratively, an edge of a first piece of material can be attached to a second piece of material, for example, to an edge of the second material piece. In certain aspects, two material pieces, which may or may not be attached, partially or wholly overlap one another in an inventive device. In a preferred embodiment, an inventive device includes a multilayered graft material, wherein the individual material layers (e.g., two, three, four, five, six, seven, eight or more material layers) are dehydrothermally and/or otherwise bonded together to form a substantially unitary graft material construct.

A piece of compliant sheet-form material used in the invention can exhibit a variety of shapes and sizes. Illustratively, an inventive device can incorporate one or more pieces of material that are generally square, rectangular or having any other suitable rectilinear shape, e.g., having three, four, five, six or any other suitable number of sides. A piece of compliant material used in the invention, or any portion thereof, can be non-rectilinear as well. Such material can have curvilinear characteristics, for example, exhibiting a generally circular or oval shape or any other suitable curvilinear shape. In some forms, a piece of compliant material has both curvilinear and non-curvilinear portions. Other suitable shapes and configurations will be recognized by those skilled in the art, and therefore, are encompassed by the present invention. In general, a compliant sheet-form material used in the invention can exhibit any suitable size and shape for use in a grafting application, for example, in repairing or otherwise treating one or more defects in a wall of a bodily structure. These include repair devices and other similar grafts that are currently known in the art, and in this regard, such devices can be suitably adapted to provide devices in accordance with the present invention.

Compliant sheet-form materials useful in the invention should generally be biocompatible, and in some advantageous embodiments of the graft devices, are comprised of a remodelable material. Particular advantage can be provided by graft devices including a remodelable collagenous material. Such remodelable collagenous materials, whether reconstituted or non-reconstituted, can be provided, for example, by collagenous materials isolated from a warm-blooded vertebrate, and especially a mammal. Such isolated collagenous material can be processed so as to have remodelable, angiogenic properties and promote cellular invasion and ingrowth. Remodelable materials may be used in this context to promote cellular growth on, around, and/or within tissue to which a grafting device of the invention is applied.

Suitable remodelable materials can be provided by collagenous extracellular matrix (ECM) materials possessing biotropic properties. For example, suitable collagenous materials include ECM materials such as those comprising submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa including small intestinal submucosa, stomach submucosa, urinary bladder submucosa, and uterine submucosa. Collagenous matrices comprising submucosa (potentially along with other associated tissues) useful in the present invention can be obtained by harvesting such tissue sources and delaminating the submucosa-containing matrix from smooth muscle layers, mucosal layers, and/or other layers occurring in the tissue source. For additional information as to submucosa useful in the present invention, and its isolation and treatment, reference can be made, for example, to U.S. Pat. Nos. 4,902,508, 5,554,389, 5,993,844, 6,206,931, and 6,099,567.

Submucosa and other ECM materials useful in the invention are preferably highly purified, for example, as described in U.S. Pat. No. 6,206,931 to Cook et al. Thus, preferred ECM materials will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of any ECM tissue used in the present invention.

A typical layer thickness for an as-isolated submucosa or other ECM tissue layer used in the invention ranges from about 50 to about 250 microns when fully hydrated, more typically from about 50 to about 200 microns when fully hydrated, although isolated layers having other thicknesses may also be obtained and used. These layer thicknesses may vary with the type and age of the animal used as the tissue source. As well, these layer thicknesses may vary with the source of the tissue obtained from the animal source.

Suitable bioactive agents may include one or more bioactive agents native to the source of the ECM tissue material. For example, a submucosa or other remodelable ECM tissue material may retain one or more growth factors such as but not limited to basic fibroblast growth factor (FGF-2), transforming growth factor beta (TGF-beta), epidermal growth factor (EGF), cartilage derived growth factor (CDGF), and/or platelet derived growth factor (PDGF). As well, submucosa or other ECM materials when used in the invention may retain other native bioactive agents such as but not limited to proteins, glycoproteins, proteoglycans, and glycosaminoglycans. For example, ECM materials may include heparin, heparin sulfate, hyaluronic acid, fibronectin, cytokines, and the like. Thus, generally speaking, a submucosa or other ECM material may retain one or more bioactive components that induce, directly or indirectly, a cellular response such as a change in cell morphology, proliferation, growth, protein or gene expression.

Submucosa or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with appropriate staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the formation of new blood vessels into the materials. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of such native bioactive components, non-native bioactive components such as those synthetically produced by recombinant technology or other methods (e.g., genetic material such as DNA), may be incorporated into an ECM material. These non-native bioactive components may be naturally-derived or recombinantly produced proteins that correspond to those natively occurring in an ECM tissue, but perhaps of a different species. These non-native bioactive components may also be drug substances. Illustrative drug substances that may be added to materials include, for example, anti-clotting agents, e.g. heparin, antibiotics, anti-inflammatory agents, thrombus-promoting substances such as blood clotting factors, e.g., thrombin, fibrinogen, and the like, and anti-proliferative agents, e.g. taxol derivatives such as paclitaxel. Such non-native bioactive components can be incorporated into and/or onto ECM material in any suitable manner, for example, by surface treatment (e.g., spraying) and/or impregnation (e.g., soaking), just to name a few. Also, these substances may be applied to the ECM material in a premanufacturing step, immediately prior to the procedure (e.g., by soaking the material in a solution containing a suitable antibiotic such as cefazolin), or during or after engraftment of the material in the patient.

Graft materials of the invention can include xenograft material (i.e., cross-species material, such as tissue material from a non-human donor to a human recipient), allograft material (i.e., interspecies material, with tissue material from a donor of the same species as the recipient), and/or autograft material (i.e., where the donor and the recipient are the same individual). Further, any exogenous bioactive substances incorporated into an ECM material may be from the same species of animal from which the ECM material was derived (e.g. autologous or allogenic relative to the ECM material) or may be from a different species from the ECM material source (xenogenic relative to the ECM material). In certain embodiments, ECM material will be xenogenic relative to the patient receiving the graft, and any added exogenous material(s) will be from the same species (e.g. autologous or allogenic) as the patient receiving the graft. Illustratively, human patients may be treated with xenogenic ECM materials (e.g. porcine-, bovine- or ovine-derived) that have been modified with exogenous human material(s) as described herein, those exogenous materials being naturally derived and/or recombinantly produced.

ECM materials used in the invention may be essentially free of additional, non-native crosslinking, or may contain additional crosslinking. Such additional crosslinking may be achieved by photo-crosslinking techniques, by chemical crosslinkers, or by protein crosslinking induced by dehydration or other means. However, because certain crosslinking techniques, certain crosslinking agents, and/or certain degrees of crosslinking can destroy the remodelable properties of a remodelable material, where preservation of remodelable properties is desired, any crosslinking of the remodelable ECM material can be performed to an extent or in a fashion that allows the material to retain at least a portion of its remodelable properties. Chemical crosslinkers that may be used include for example aldehydes such as glutaraldehydes, diimides such as carbodiimides, e.g., 1-ethyl-3-(3-dimethylaminopropyl)carbodiimide hydrochloride, ribose or other sugars, acyl-azide, sulfo-N-hydroxysuccinamide, or polyepoxide compounds, including for example polyglycidyl ethers such as ethyleneglycol diglycidyl ether, available under the trade name DENACOL EX810 from Nagese Chemical Co., Osaka, Japan, and glycerol polyglycerol ether available under the trade name DENACOL EX 313 also from Nagese Chemical Co. Typically, when used, polyglycerol ethers or other polyepoxide compounds will have from 2 to about 10 epoxide groups per molecule.

Turning now to a discussion of drying techniques that can be useful in certain embodiments of the invention, drying by evaporation, or air drying, generally comprises drying a partially or completely hydrated remodelable material by allowing the hydrant to evaporate from the material. Evaporative cooling can be enhanced in a number of ways, such as by placing the material in a vacuum, by blowing air over the material, by increasing the temperature of the material, by applying a blotting material during evaporation, or by any other suitable means or any suitable combination thereof. The amount of void space or open matrix structure within an ECM material that has been dried by evaporation is typically more diminished than, for example, an ECM material dried by lyophilization as described below.

A suitable lyophilization process can include providing an ECM material that contains a sufficient amount of hydrant such that the voids in the material matrix are filled with the hydrant. The hydrant can comprise any suitable hydrant known in the art, such as purified water or sterile saline, or any suitable combination thereof. Illustratively, the hydrated material can be placed in a freezer until the material and hydrant are substantially in a frozen or solid state. Thereafter, the frozen material and hydrant can be placed in a vacuum chamber and a vacuum initiated. Once at a sufficient vacuum, as is known in the art, the frozen hydrant will sublime from the material, thereby resulting in a dry remodelable material.

In alternative embodiments, a hydrated ECM material can be lyophilized without a separately performed pre-freezing step. In these embodiments, a strong vacuum can be applied to the hydrated material to result in rapid evaporative cooling which freezes the hydrant within the ECM material. Thereafter, the frozen hydrant can sublime from the material thereby drying the ECM material. Desirably, an ECM material that is dried via lyophilization maintains a substantial amount of the void space, or open matrix structure, that is characteristic of the harvested ECM material.

Drying by vacuum pressing generally comprises compressing a fully or partially hydrated remodelable material while the material is subject to a vacuum. One suitable method of vacuum pressing comprises placing a remodelable material in a vacuum chamber having collapsible walls. As the vacuum is established, the walls collapse onto and compress the material until it is dry. Similar to evaporative drying, when a remodelable material is dried in a vacuum press, more of the material's open matrix structure is diminished or reduced than if the material was dried by lyophilization.

In certain aspects, the invention utilizes graft materials that include a multilaminate material. Such multilaminate materials can include a plurality of ECM material layers bonded together, a plurality of non-ECM materials bonded together, or a combination of one or more ECM material layers and one or more non-ECM material layers bonded together. To form a multilaminate ECM material, for example, two or more ECM segments are stacked, or one ECM segment is folded over itself at least one time, and then the layers are fused or bonded together using a bonding technique, such as chemical cross-linking or vacuum pressing during dehydrating conditions. An adhesive, glue or other bonding agent may also be used in achieving a bond between material layers. Suitable bonding agents may include, for example, collagen gels or pastes, gelatin, or other agents including reactive monomers or polymers, for example cyanoacrylate adhesives. As well, bonding can be achieved or facilitated between ECM material layers using chemical cross-linking agents such as those described above. A combination of one or more of these with dehydration-induced bonding may also be used to bond ECM material layers to one another.

A variety of dehydration-induced bonding methods can be used to fuse together portions of an ECM material. In one preferred embodiment, multiple layers of ECM material are compressed under dehydrating conditions. In this context, the term “dehydrating conditions” is defined to include any mechanical or environmental condition which promotes or induces the removal of water from the ECM material. To promote dehydration of the compressed ECM material, at least one of the two surfaces compressing the matrix structure can be water permeable. Dehydration of the ECM material can optionally be further enhanced by applying blotting material, heating the matrix structure or blowing air, or other inert gas, across the exterior of the compressed surfaces. One particularly useful method of dehydration bonding ECM materials is lyophilization.

Another method of dehydration bonding comprises pulling a vacuum on the assembly while simultaneously employing the vacuum to press the assembly together. Again, this method is known as vacuum pressing. During vacuum pressing, dehydration of the ECM materials in forced contact with one another effectively bonds the materials to one another, even in the absence of other agents for achieving a bond, although such agents can be used while also taking advantage at least in part of the dehydration-induced bonding. With sufficient compression and dehydration, the ECM materials can be caused to form a generally unitary ECM structure.

It is advantageous in some aspects of the invention to perform drying and other operations under relatively mild temperature exposure conditions that minimize deleterious effects upon any ECM materials being used, for example native collagen structures and potentially bioactive substances present. Thus, drying operations conducted with no or substantially no duration of exposure to temperatures above human body temperature or slightly higher, say, no higher than about 38° C., will preferably be used in some forms of the present invention. These include, for example, vacuum pressing operations at less than about 38° C., forced air drying at less than about 38° C., or either of these processes with no active heating—at about room temperature (about 25° C.) or with cooling. Relatively low temperature conditions also, of course, include lyophilization conditions.

As well, graft materials useful in the invention may be comprised of biocompatible materials derived from a number of biological polymers, which can be naturally occurring or the product of in vitro fermentation, recombinant genetic engineering, and the like. Purified biological polymers can be appropriately formed into a substrate by techniques such as weaving, knitting, casting, molding, and extrusion. Suitable biological polymers include, without limitation, collagen, elastin, keratin, gelatin, polyamino acids, polysaccharides (e.g., cellulose and starch) and copolymers thereof.

Graft devices of the invention can also include a variety of synthetic polymeric materials including but not limited to bioresorbable and/or non-bioresorbable plastics. Bioresorbable, or bioabsorbable polymers that may be used include, but are not limited to, poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyhydroxyalkanaates, polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, and polyphosphazenes. These or other bioresorbable materials may be used, for example, where only a temporary blocking or closure function is desired, and/or in combination with non-bioresorbable materials where only a temporary participation by the bioresorable material is desired.

Non-bioresorbable, or biostable polymers that may be used include, but are not limited to, polytetrafluoroethylene (PTFE) (including expanded PTFE), polyethylene terephthalate (PET), polyurethanes, silicones, and polyesters and other polymers such as, but not limited to, polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; and rayon-triacetate.

Turning now to a more detailed discussion of resilient elements useful in the present invention, it will be understood that a resilient element occurring in a given inventive device can comprise one or more individual pieces of material or other objects (e.g., pieces of resilient wire). Although not necessary to broader aspects of the invention, when a resilient element includes a multitude of components, a particular component may be attached or otherwise joined to any or all of the other components. In some cases, a device includes two or more resilient element components that are not joined to one another yet collectively enable the device to exhibit qualities (e.g., performance and/or handling characteristics) in accordance with the present invention. In one embodiment, a device includes at least two resilient elements that cooperate with one another in an essentially controlled manner to provide a desirable arrangement for delivery into the body and deployment upon delivery.

Although not necessary to broader aspects of the invention, in general, a resilient element, when retained in association with a compliant sheet-form material, will exhibit a relaxed or non-deformed condition effective to present the sheet-form material, or at least a segment thereof, in a generally planar form. In some aspects, however, a similarly non-deformed resilient element will be effective to present the compliant sheet-form material in a form where no portion of the material is planar. Illustratively, an inventive grafting device can be adapted so that when the resilient element is in a relaxed condition, the associated compliant sheet-form material is presented in a form having curvilinear and/or other suitable non-planar qualities.

While associated with the sheet-form material, the resilient element can be deformed (e.g., folded, rolled, twisted, etc.) to alter the shape of the overall device. For example, the shape of an inventive device can be transformed in this manner so that the device occupies a space (e.g., a volume within a delivery device lumen) in which the device would not have been able to fit prior to the transformation. In some forms, a compacted or otherwise transformed device is positioned in a delivery device lumen, wherein the device is constrained by an interior wall of the device and substantially maintained in this transformed condition. In an arrangement of this sort, the constrained device should still be movable in the delivery device lumen for removing the device from the lumen during delivery. When the device is compacted, the compliant sheet-form material deforms along with the deformed resilient element, either in a somewhat controlled or random fashion. The compliant material can deform in any suitable manner including some that involve portions of the material being folded and/or rolled. And in this regard, upon removing the grafting device from the delivery device lumen, the resilient element, as it generally returns to its relaxed condition, is effective to spread open the associated compliant material or otherwise essentially return the material to its prior, non-deformed shape.

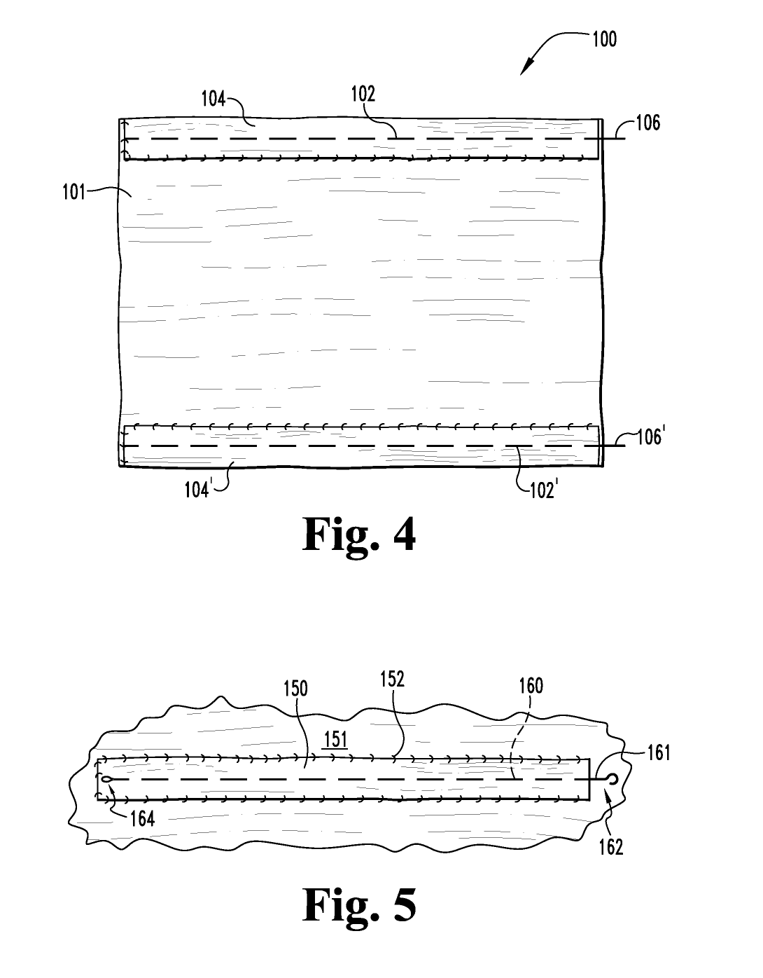

A resilient element useful in the present invention can incorporate one or more individual resilient objects. Referring now to FIG. 4, shown is a grafting device 100 in accordance with another embodiment of the present invention. Grafting device 100 includes a piece of compliant sheet-form material 101, a first resilient element 102, and a second resilient element 102′. First resilient element 102 and second resilient element 102′ are removably positioned in a first receiving area 104 and a second receiving area 104′, respectively. When so positioned, first resilient element 102 and second resilient element 102′ are effective to present sheet-form material 101 in a generally planar form as shown in FIG. 4. In this particular embodiment, opposing edges of material piece 101 are folded over and sutured to form the receiving areas. More particularly, sutures extend along the side edge and one of the ends of each folded portion, leaving open ends into which the resilient elements can be received. First resilient element 102 and second resilient element 102′ include first retrieving portion 106 and second retrieving portion 106′, respectively, which each have a generally straight end and extend a distance away from material piece 101. By extending away from the material in this manner, the retrieving portions are potentially easier to locate and retrieve in the body following initial deployment.

A resilient element useful in the present invention can be retained in association with a compliant sheet-form material in a variety of manners including some that involve directly attaching a resilient element to a graft material and some that do not. In certain forms, a resilient element is reversibly attached to a sheet-form material in such a manner that the two can be detached when so desired. This sort of attachment can be accomplished in a variety of fashions including some that involve the use of single- or multiple-part coupling devices that permit decoupling; bonding between components that can be reversed or otherwise broken when desired; and other suitable means of reversibly attaching two objects together as known by those skilled in the art.

As well, the present invention provides a numbers of devices where a resilient element is retained in association with a sheet-form material without being attached to the material. Illustratively, a resilient element can be positioned in a receiving area occurring along the material, for example, in a substantially confined space occurring along a peripheral or other region of a material piece. Such a receiving area may be in the form of a single- or multiple-part sleeve, pocket, channel or other similar adaptation in which a resilient element useful in the invention can be positioned and retained. A receiving area of this sort may be defined in whole or in part by the sheet-form material itself. For example and referring again to FIGS. 1 and 4, a suitable receiving area can be provided by a folded peripheral region of a piece of material. Additionally or alternatively, a resilient element can be woven through a material piece one or more times for retainment purposes. As will be appreciated by those skilled in the art, peripheral and/or non-peripheral regions of a piece of material can be manipulated in a variety of manners to provide one or more receiving areas finding use in the present invention. In some cases, a suitable receiving area is formed by a portion of material that has been folded and/or rolled around, through, over, etc. another material portion, and fixed (e.g., glued, sutured, stapled, etc.) to this other portion to maintain the receiving area. In other cases, a receiving area is maintained without using such additional components.

In certain embodiments, one or more objects that are initially separate from a compliant sheet-form material are combined with the material to provide all or part of a receiving area in which a resilient element can be positioned and retained. Suitable objects for this purpose include but are not limited to pieces of material (e.g., tubes, sleeves, bands, etc.), staples, suture material and other similar retaining elements that can be joined with the material to alone, or in conjunction with one or more other objects, provide a receiving area. With reference now to FIG. 5, shown is a segment of material 150 that is sutured to a compliant sheet-form material 151 to provide a receiving area in which a resilient element 160 can be positioned. More particularly, a plurality of sutures 152 extend along both side edges and one of the ends of the segment, leaving an open end into which the resilient element can be received. Material segments of this sort can be used in conjunction with any of the sheet-form materials described herein, and can be placed at any suitable location on a given piece of material including peripheral and/or non-peripheral locations. Further, while the pieces of material in the current embodiment are secured to one another with sutures 152, the two could be secured in any suitable manner, e.g., with an adhesive.

When a resilient element used in the invention has an end, this end can be configured in a variety of fashions. For example, the resilient element shown in FIG. 1 has one end that is generally straight and another end (extending from the material) that has a looped tip. Alternatively, both of the resilient elements shown in FIG. 4 have ends that are generally straight. Referring again to FIG. 5, resilient element 160 includes a retrieving portion 161 that extends a distance from material segment 150, and has a hooked tip 162. The opposite end of resilient element 160 includes a looped tip 164. Such a looped tip can prevent damage to sheet-form material 151 and/or segment of material 150 as resilient element 160 traverses the receiving area. A tip of this sort can also prevent damage to patient tissue during removal of resilient element 160 from the body following initial deployment of the device. Other similar adaptations for preventing such damage will be recognized by those skilled in the art, and are therefore encompassed by the present invention. Additionally, the receiving area depicted in FIG. 5 and some of the other receiving areas described herein can be adapted so that a resilient element can be passed through one or more ends or other openings of the receiving area.

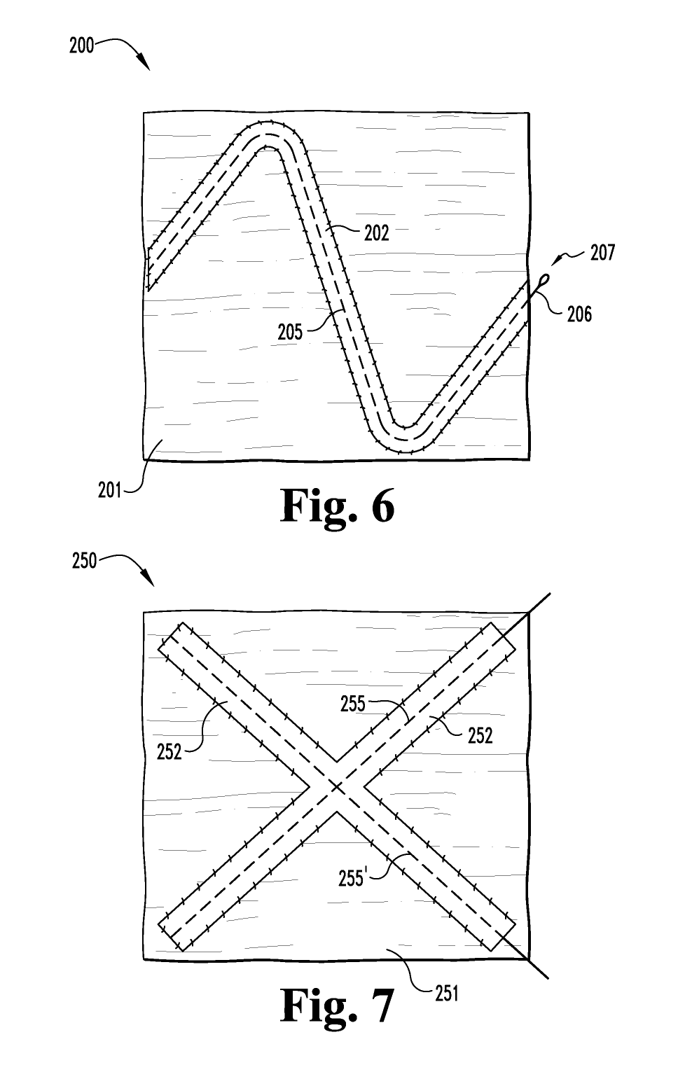

A resilient element useful in the invention can be shaped and configured in a variety of manners, and can occur at any suitable location along a piece of graft material, for example, along peripheral and/or non-peripheral regions of a material piece. With reference now to FIG. 6, shown is a grafting device 200 according to another embodiment of the present invention. Grafting device 200 includes a compliant sheet-form material 201 and a material segment 202 sutured thereto to provide a receiving area in which a resilient element 205 can be positioned as generally shown. A plurality of sutures extend along portions of the perimeter of material segment 202 to provide a receiving area open end into which resilient element 205 is received. Resilient element 205 includes a retrieving portion 206, which extends from this open end, and has a looped tip 207. Shown in FIG. 7 is a grafting device 250 according to yet another embodiment of the present invention. Grafting device 250 includes a compliant sheet-form material 251 and an X-shaped material segment 252 sutured thereto to provide receiving areas in which a first resilient element 252 and a second resilient element 252′ can be received as generally shown. These resilient elements, which are not attached to one another, can translate over one another during placement and removal.

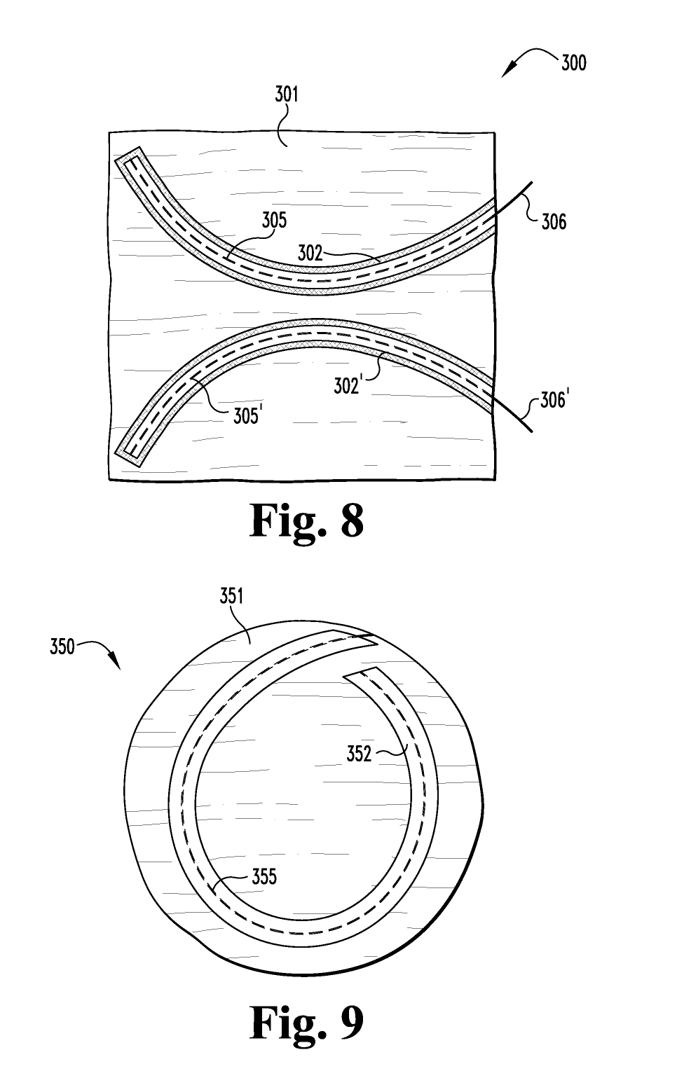

As discussed elsewhere herein, single- and multi-layered graft materials find use in the present invention. In some cases, an inventive device includes multiple layers of compliant material, wherein a resilient element, when retained in association with the material, resides wholly or partially between any two material layers. In certain embodiments, a resilient element is positioned in an at least somewhat defined channel or other similar receiving area occurring between two material layers. Illustratively and referring now to FIG. 8, shown is grafting device 300 according to another embodiment of the present invention. Grafting device 300 includes a compliant sheet-form material 301 formed with multiple material layers. Device 300 also includes a first resilient element 305 and a second resilient element 305′ removably positioned in a first receiving area 302 and a second receiving area 302′, respectively. Although not necessary to broader aspects of the invention, the receiving areas both exhibit a generally curvilinear shape along the material piece. Receiving areas of this sort can be shaped and configured in a variety of manners, and can occur at any suitable location along a material.

First receiving area 302 and second receiving area 302′ occur between overlapping material layers. Portions of the overlapped material layers can be bonded together in the patterns shown to form receiving channels through which the resilient elements can be passed and retained. Any suitable form of bonding can be used including those involving adhesives, compression, dehydration, heating, etc. In some cases, one or more receiving areas are formed by suturing together overlapped material portions or otherwise securing one material layer to another material layer in a manner that provides a space through which a resilient element can be passed and retained. First resilient element 305 and second resilient element 305′ include first retrieving portion 306 and second retrieving portion 306′, respectively, which each have a generally straight end and extend a distance away from material piece 301. Such retrieving portions are optional as with any of the other device embodiments described herein.

With reference now to FIG. 9, shown is a grafting device 350 according to another embodiment of the present invention. Grafting device 350 includes a piece of compliant sheet-form material 351 and a segment of material 352 attached to the material to provide a generally circular receiving area for a resilient element along the material. Sheet-form material 351 exhibits a generally circular shape as well, although other suitably shaped materials may also be used. A resilient element 355 is positioned in the receiving area with a small portion of the element extending from one end of the receiving area. In some forms, such a resilient element is adapted so that it can fit entirely within the receiving area.

A resilient element useful in the invention can be formed with one or more of a variety of materials. In this regard, many suitable materials exhibiting resiliency, and many suitable resilient objects, are know to those skilled in the art, and are therefore encompassed by the present invention. In general, a suitable resilient element will be one having qualities enabling it to behave as described herein. Materials useful in some embodiments include gold, rhenium, platinum, palladium, rhodium, ruthenium, various stainless steels, tungsten, titanium, nickel, cobalt, tantalum, iron, and copper, as well as alloys of these and other suitable metals, e.g., cobalt alloys, such as Elgiloy®, a cobalt-chromium-nickel alloy, MP35N, a nickel-cobalt-chromium-molybdenum alloy, and a nickel-titanium alloy, e.g., Nitinol®. Additionally or alternatively, resilient elements can include material in the form of yarns, fibers, and/or resins, e.g., monofilament yarns, high tenacity polyester, and the like, as well as other plastic, resin, polymer, woven, and fabric surgical materials, other conventional synthetic surgical materials, such as shape-memory plastics, and combinations of such materials.

All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

1. A grafting device deliverable into the body for repairing a defect in a wall of a bodily structure, the grafting device comprising:
a compliant sheet-form material; and
a removable resilient element retained in association with the sheet-form material and exhibiting a relaxed condition effective to present at least a segment of the sheet-form material in a generally planar form, the resilient element deliverable in its entirety into the body and having an end that extends beyond an outermost edge of the sheet-form material and that can be grasped for disassociating the resilient element from the sheet-form material for removing the resilient element from the body.

2. The grafting device of claim 1, wherein the sheet-form material is comprised of a remodelable material.

3. The grafting device of claim 1, wherein the sheet-form material is comprised of an extracellular matrix material.

4. The grafting device of claim 3, wherein the extracellular matrix material comprises submucosa, serosa, pericardium, dura mater, peritoneum, or dermal collagen.

5. The grafting device of claim 1, wherein the sheet-form material is comprised of a synthetic polymeric material.

6. The grafting device of claim 1, wherein the resilient element is comprised of a metallic material.

7. The grafting device of claim 1, wherein the resilient element is comprised of a synthetic polymeric material.

8. The grafting device of claim 1, wherein the resilient element is deformable to a deformed condition for positioning the grafting device in a delivery device lumen for delivery into the body.

9. The grafting device of claim 1, wherein the resilient element is positioned in a receiving area occurring along the sheet-form material.

10. The grafting device of claim 9, wherein the receiving area occurs along a peripheral region of the sheet-form material.

11. The grafting device of claim 9, further comprising one or more material segments joined with the sheet-form material to provide the receiving area.

12. The grafting device of claim 9, further comprising suture material joined with the sheet-form material to provide the receiving area.

13. The grafting device of claim 9, wherein a folded peripheral region of the sheet-form material provides the receiving area.

14. The grafting device of claim 9, wherein said receiving area comprises a channel through which the resilient element can pass for disassociating the resilient element from the sheet-form material.

15. The grafting device of claim 1, wherein the sheet-form material comprises a single-layer material.

16. The grafting device of claim 1, wherein the sheet-form material comprises two or more layers of material.

17. The grafting device of claim 16, wherein the resilient element is positioned at least partly between two of said two or more layers of material for retaining the resilient element in association with the sheet-form material.

18. An apparatus for delivering a grafting device into the body and thereafter removing one or more elements of the grafting device from the body, the apparatus comprising:
a delivery device having a lumen communicating with a distal end opening, the distal end opening configured for passage into the body; and
a grafting device positioned in the delivery device lumen and effective to repair a defect in a wall of a bodily structure, the grafting device comprising:
a compliant sheet-form material; and
a removable resilient element retained in association with the sheet-form material, the resilient element deliverable in its entirety into the body and having an end that can be grasped for disassociating the resilient element from the sheet-form material for removing the resilient element from the body, wherein the resilient element exhibits a deformed first condition when the grafting device is positioned in the delivery device lumen, and is adapted to attain a second condition when the grafting device is removed from the delivery device lumen, the second condition effective to present at least a segment of the sheet-form material in a generally planar form in the body for placement at the bodily structure wall defect.

19. The apparatus of claim 18, wherein said resilient element is received within a channel for retaining the resilient element in association with said sheet-form material.

20. The apparatus of claim 19, wherein said end extends beyond an outermost edge of the sheet-form material

21. A method for delivering a grafting device into the body, the method comprising:
providing a delivery device having a lumen communicating with a distal end opening, the distal end opening configured for passage into the body;
providing a grafting device positioned in the delivery device lumen and effective to repair a defect in a wall of a bodily structure, the grafting device comprising:
a compliant sheet-form material; and
a removable resilient element retained in association with the sheet-form material and having an end that can be grasped for disassociating the resilient element from the sheet-form material for removing the resilient element from the body, wherein the resilient element exhibits a deformed first condition when the grafting device is positioned in the delivery device lumen;
positioning the delivery device distal end opening in the body; and
removing the grafting device from the delivery device lumen through the distal end opening, wherein the resilient element is delivered in its entirety into the body and attains a second condition effective to present at least a segment of the sheet-form material in a generally planar form for placement at the bodily structure wall defect.
